(19) United States
(12) Patent Application Publication
(10) Pub. No.: US 2020/0360673 A1
(43) Pub. Date: Nov. 19, 2020

(54) ENCASING DEVICE FOR DISPENSING TOPICAL PRODUCTS FOR ORAL, VULVAR AND ANAL USE, ATTACHABLE TO PORTABLE APPLICATOR FOR VAGINAL AND RECTAL USES

(71) Applicant: Doris Hexsel, Porto Alegre (BR)

(72) Inventor: Doris Hexsel, Porto Alegre (BR)

(21) Appl. No.: 16/643,091

(22) PCT Filed: Jul. 13, 2018
(86) PCT No.: PCT/BR2018/050238

§ 371 Date: Feb. 28, 2020

(30) Foreign Application Priority Data
Jul. 27, 2017 (BR) BR 102017016174-9

(51) Int. Cl. A61M 31/00; A61M 35/00

(52) U.S. Cl. CPC A61M 31/00; A61M 35/003; A61M 2210/0625; A61M 2210/1067; A61M 2210/1475

(57) ABSTRACT

Set of applicators for dispensing topical products in a single dose in the anal, oral, rectal, vaginal and vulvar mucous and semi-mucous membranes. The encasement type packaging is similar to a condom, but smaller, presenting a compartment filled with the desired pharmaceutical formulation to be used in a single dose, located at its distal end, which contains orifices through which the product will be expelled. The content of the packaging can be applied with the finger of an individual or with a portable applicator. Said portable applicator does not directly contact the mucous and semi-mucous membranes, since it is covered by the fine encasement. The applicator has two encasable parts, making the transport thereof efficient, since it is extended upon encasement. A rounded anatomic part is encased in the applicator for compressing the product reservoir. The solution developed can be used to deliver medications to treat medical conditions and illnesses, or promote relief and comfort, in a safe, efficient, practical and discrete manner.

### TECHNOLOGICAL FIELD OF THE INVENTION

The present invention generally pertains to the technological sector of systems for deliver a pharmaceutical compositions and more specifically refers to a novel arrangement composed of a fine disposable encasement for dispensing products in a single dose in the oral, vaginal, vulvar anal and rectal mucous and semi-mucous membranes. Additionally, said encasement may also be attached to a portable applicator for dispensing medications into the vaginal and rectal cavities; and, if the need arises to reach such depth, the user's finger can be used to deliver medications in the oral, vulvar and anal cavities.

### KNOWN STATE OF THE ART

Topical applications of pharmaceutical formulations in the mouth, vulva or anus are generally done directly with the fingers, with the potential risk of contamination. Yet formulations for vaginal or rectal use are done with the assistance of vaginal or rectal applicators.

It is known that women are more prone to presenting diseases in the genital region than men, due to the anatomy of the feminine body. Vaginal applicators are used for dispensing pharmaceutical preparations in the uppermost region of the vagina in order to treat diseases or conditions affecting the feminine urogenital system, cervix, uterus, ovaries, fallopian tubes and peritoneal cavity. They can also be used to administer contraceptives and lubricants or other formulations in the vagina or in the rectal ampulla.

Vaginal and rectal applicators of various formats have been used for decades, yet they were originally designed to administer vaginal contraceptives to the cervix and vaginal creams. Conventional applicators found on the market present a standard size, measuring some centimeters in length, which is considered large and inconvenient for purposes, transport, and use, due to the fact that women prefer discretion, especially with certain formulations, such as lubricants.

A large quantity of medications needs to be applied once or twice per day. Most applicators used for this present a long and rigid format, measure around 12 cm, have a caliber of 1 cm or larger, and their reservoirs need to be filled with the formulation whenever they need to be used. This implies that, at times, the user needs to carry one or more applicators and, further, the pharmaceutical formulation for the professional environment or on trips and moreover needs to appropriately discard the packaging, generally plastic and rigid. The commercial presentations for vaginal use creams today include a certain number of disposable applicators to satisfy the content of the tube of the product to be used. The same is seen in relation to rectal applicators.

Known applicators are accompanied by a tube containing the formulation and the tip thereof should be placed inside the applicator to fill it with the dose needed for each use. Firstly, the nozzle of the tube should be perforated with the under part of the lid and, afterwards, the applicator should be threaded/attached to the tube in the place of the lid. The tube should then be squeezed so that the applicator is filled with the necessary amount of the product. Thereafter, the applicator should be removed from the tube and it should be immediately closed with the lid. Other applicators are designed with a special end to dispense an egg at the bottom of the vagina, which will come apart and release the content at this point.

Some more recent applicators are already filled with the formulation and ready to be used, yet some packaging needs to be cut and offers difficulties in terms of their format for introduction into the vagina. Additionally, they entail a higher cost for the product and inadequate disposal, since they are usually discarded in wastepaper baskets in restrooms. Some products for masculine use already contain genital lubricants, as is the case of lubricated condoms.

One problem associated to the current state of the art is that the market does not have practical packaging for lubricants that can be used and carried discretely, quickly and comfortably by women or men. They always have to depend on carrying long and rigid applicators, disposable or on a tube of the chosen lubricant, requiring a more lengthy and complex procedure to use and adequately use and dispose it.

Oftentimes the same product used in the vulva or anus needs to be applied in the vaginal and rectal regions as well, with the objective of treating problems in these regions.

Therefore, there is a need for a device that can be used in a versatile manner, both in the mouth, vulva and anus, as well as the vagina and the rectum. There is no packaging on the market that contains a reservoir filled with a single dose pharmaceutical formulation, disposable, sterile, practical, malleable and of reduced size, that can be used in the mouth, vulva, vagina, anus and rectum, being attached to the user's finger or to a portable applicator, that offers safety and protection to the user.

Having said this, it is noted that there are some patent documents that describe the current state of the art, such as patent of invention no CN201823159, entitled “Vaginal Applicator”. This discloses a vaginal applicator that comprises a sleeve and is characterized in that a sterilized cotton pad is disposed on the inner surface of the front section of the sleeve, an elongation bar is disposed on the sleeve and a medication groove is disposed on the front part of the bar. Additionally, it has the advantage of preventing cross infection and diminishing the work hardship of medical personnel.

Patent of invention no US20040044328, entitled “Applicator and stimulator device”, describes an applicator and stimulator device which comprises a flexible sleeve. The inside of the sleeve is adapted to house a human finger. This sleeve is closed at one end and presents one or more cells on its outer wall. Said cells contain fluids retained by sealing. The sealing can be open or is susceptible to breakage when pressure is applied through the wall or the wall is in contact with an environment that weakens the sealing. The sleeve is formed from an elastic material such as natural rubber or a synthetic, such as a highly plasticized PVC resin. The cells associated with the outer wall are formed by creating depressions in the wall during the molding of the device. In its non-flexible state of the body of the sleeve it is substantially cylindrical and the cells are preferably formed on a portion of the surface of the sleeve near the closed and supporting end at an angle of about 90 degrees on the surface measured from the axis of the cylinder. The cells may contain a lubricant, a pharmacologically active compound or a combination of such substances. The preferred pharmacologically active compound is a vasoactive compound either alone or in a mixture with one or more adjuvants to assure the consistency and retention, when applied to the vasodilating tissue. At least a part of the periphery of the open end of the sleeve may have a closed channel, filled with a lubricant which is released when the seal is ruptured.

Patent of invention no PI 0602169-7, entitled “Applicator for a Product”, describes an applicator device made of a first glove that is substantially open at the top and at the bottom. A product support is received inside the first glove, this product support comprising a second glove. The lower part of the second glove may be contacted by the finger of a person to adjust the second glove inside the first glove and temporarily jam the second glove in the first glove. On the inner surface of the first glove and the outer surface of the second glove are cooperating structures to keep the second glove in a given position in the first glove by pressing the first glove. The cooperating structure may be a cooperating serrated structure on each glove, a serrated structure on a glove and a locking protrusion on the other glove or any other interference on each glove. In one embodiment there may be one or more openings in the first glove to facilitate the flexing of the first glove in contact with the second glove and the temporary jamming of the second glove in the first glove. The product may be a cosmetic product that includes deodorants and antiperspirants. In use the second glove is adjusted inside the first glove by a pushing action on the bottom of the second glove. The sides of the first glove are grasped in a pressure application action that retains the second glove inside the first glove during application of the product. By relaxing the pressing action, the second glove may recoil inwardly of the first glove and a lid applied.

Lastly, patent of invention no PI 0112396-3, entitled “Tampon Applicator having Improved Finger Grasp”, describes useful applicators for inserting objects into cavities of the body, particularly catamenial and prophylactic devices in a vaginal channel. The applicators comprise an insertable tubular element and a back-fit grasping element. The grasping element has an outer diameter greater than that of the inserting element. An interface is created due to this difference in size, which provides a visual or tactile attraction having the applicator insertion depth, and afterwards positions the object inside the cavity of the body. The grasping element comprises at least an elevated area on its surface disposed externally, providing resistance to the movement of the finger of the user's hand in response to longitudinal forces in the inserting element. Methods of assembling the applicators are also described.

Analogously, patent of invention no US20030088217, entitled “Applicator for the delivery of topical formulations into mucosal cavities” also describes an applicator. This invention refers to a vaginal/anal-rectal applicator for the uniform administration of all topical formulations for treating and/or preventing any infection and/or abnormal conditions of mucous cavity caused by any pathogenic agent and/or disease. The applicator comprises a body which extends longitudinally which has proximal and distal ends. The proximal end is located near the external site of a mucous cavity accessible to a user. The body has external perforations, made with a series of grooves or orifices, for uniform distribution of any formulation to be delivered to the cavity of the mucous membranes of the user. After inserting the applicator and expelling the formulation into the mucous cavity, the formulation, which is contained in a reservoir, moves through a diffusion channel which has a small volume, before being expelled through the perforations.

With this, in light of all the drawbacks existing in the oral, vaginal, vulvar, rectal and anal applicators currently used and described in the state of the art, there is a visible gap for creating an object capable of storing a desired pharmaceutical formulation in a single dose to be dispensed with the assistance of the user's finger or a portable applicator, whereby enabling the application of a formulation in a hygienic, practical, discrete and comfortable manner.

### NOVELTIES AND OBJECTIVES OF THE INVENTION

With the aim of providing substantial improvements and solutions for the drawbacks cited in the state of the art, the present invention proposes an encasement that consists of a single dose packaging, to be optionally attached to a portable applicator and an encasement part, to dispense products in a safe manner into the mucous and semi-mucous membranes of oral, vulvar, vaginal, anal and rectal regions.

The encasement with packaging function proposed in this document is similar to a condom, yet smaller in size and width. It presents a compartment filled with a pharmaceutical formulation to be used in a single dose, located at its distal end. The content of the packaging can be applied with the user's finger in the oral, vulvar and anal regions or with a portable applicator, of reduced size, when the use is intended for the vaginal and rectal regions.

The solution developed can be used to dispense different specific pharmaceutical formulations in the regions cited above, with the objective of treating medical conditions and diseases, or promoting relief and comfort, as is the case of patients which require lubrication and hydration of these sites.

The invention also has two other devices: a portable elongated applicator of reduced and discrete size, similar to a finger, which presents the purpose of dispensing the formulation inside the vaginal or rectal cavity, and an anatomic part which attaches to the upper part of the portable applicator and helps dispense the formulation inside the vaginal or rectal cavity.

Accordingly, the present invention provides an encasement for a product in a single dose intended for person use in the oral, vulvar, vaginal, anal and rectal regions, with the objective of remedying a discomfort, clinical condition or disease, bringing practicality, comfort, discretion, safety and hygiene to the user.

### DESCRIPTION OF THE ACCOMPANYING DRAWINGS

For the present invention to be fully understood and put into practice by any technician from the technology sector, it has to be described in a clear, concise and sufficient manner, based on the accompanying drawings, which illustrate and support it, as indicated below:

FIG. 1 represents the encasement in an unrolled/wrapped format from a front perspective.

FIG. 2 represents the encasement in elongated format from a front perspective.

FIG. 3 represents the two unencased parts that make up the portable applicator from a front perspective.

FIG. 4 represents the encased portable applicator from a front perspective.

FIG. 5 represents the anatomical encasement part from a front perspective.

FIG. 6 represents the combination formed by the packaging, portable applicator and anatomical encasement part from a front perspective.

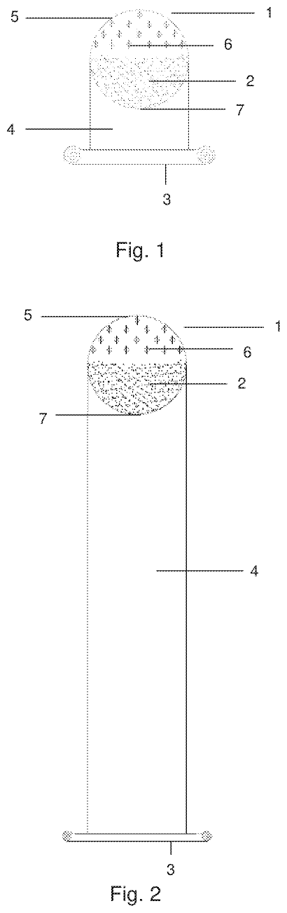

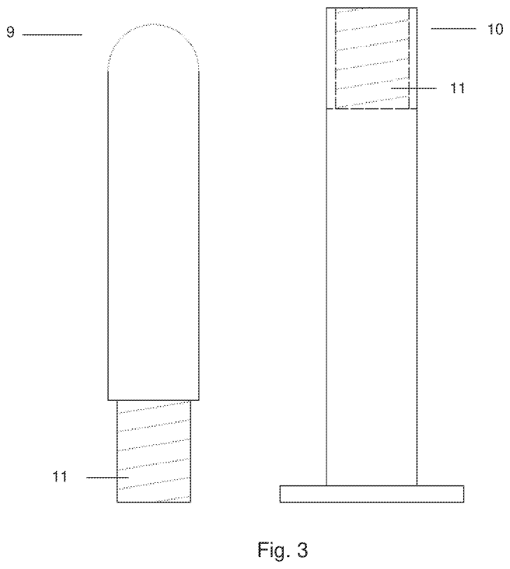

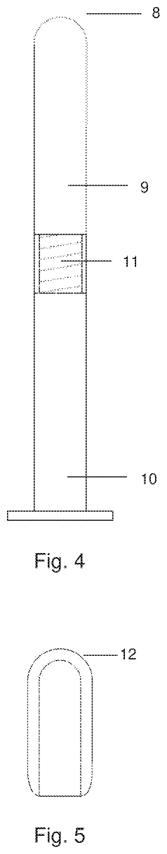

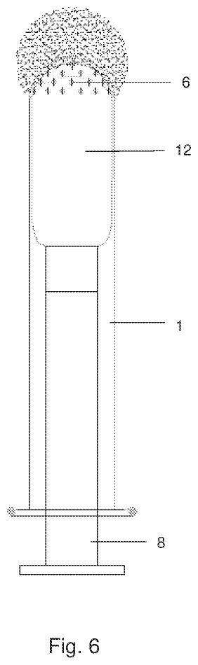

### DESCRIPTION

The present invention consists of a single dose packaging type encasement (01) for dispensing formulations in a safe manner into the oral, vulvar, vaginal, anal and rectal mucous and semi-mucous membranes, being further endowed with two devices (08)(12) to facilitate vaginal and rectal application. Said devices (08)(12) do not come into contact with the mucous and semi-mucous membranes and are reusable, since they are covered by packaging (01) that comes into direct contact with said regions and is disposable.

In short, the invention is based on the search for a form of applying formulations in the oral, vulvar, vaginal, anal and rectal mucous and semi-mucous membranes in a safe manner, easy to carry in bags and vanity cases by being low volume, and low in cost.

The present invention refers to the following objects: (I) malleable packaging type encasement (01) having a compartment (02) for the desirable formulation, (II) portable cylindrical elongatable applicator (08) and (III) encasement part (12) having an anatomic design.

Said encasement (01) is kept in its unrolled format until the moment of use, similar to widely marketed male condoms. This is in fact a refill of the pharmaceutical formulation used. The lower end (03) of the encasement (01) is open to be placed on the portable applicator or on the very finger of the user, and the lower end (03) is further endowed with a hardened fine ring on which the encasement is wrapped/rolled. The encasement also presents a cylindrical body (04) and upper end in semi-spherical format (05). Its distal portion, located at the upper end, contains its own compartment (02) to store the desired or chosen formulation. This distal portion also presents orifices (06) through which the product is dispensed when the compartment (02) is compressed by a finger or by the portable cylindrical elongatable applicator (08) and ruptured.

The inner part of the encasement is composed of a small thimble (07) made of latex, malleable rubber or similar, which holds the compartment (02) in the upper end (05) of the encasement (01), that enables the product of the compartment (02) to be pushed outwardly the orifices (06) thereof, with the help of the portable applicator and of the rubber part, or even the user's finger. The small thimble (02) is designed to resist to the pressure of the applicator or user's finger while the compartment (02) is designed to suffer rupture of the membrane containing the medicine and release said medicine through the said orifices (06). The packaging type casing (01) is disposable and should not be reused. The compartment (02) and the amount of product to be dispensed may vary according to the purpose of use and the type of formula to be used. Equally, the orifices (06) may vary in form and size, according to different products. Accordingly, the compartment (02) which contains the product to be dispensed through the orifices (06) may have its volume altered to satisfy specific purposes.

When the small thimble (07) suffers a pressure made by a finger or an elongatable applicator, the compartment (02) is pushed toward the orifices (06). With this pressure, the compartment (02) is ruptured and releases the product by the orifices into the cavity intended to receive the medicine. Although the compartment (02) is ruptured, the small thimble (07) resists to the pression and no product is spilled by this membrane into the finger or the enlongatable applicator, thus avoiding contamination of the product. For this feature the compartment (02) membrane may be thinner than the small thimble (07) or otherwise, be made of a material less resistant to the said pressure applied. Because of that, the product is fully released through the orifices (06) into the mucosa, avoiding waste of the product. This is achieved because the small thimble (07) fills the entire contour of the upper end (05) with the pressure and its structure is kept intact, preventing the product to leek to the finger or to the portable cylindrical elongatable applicator (08) that is making the pressure.

The packaging type encasement (01) is commercialized inside protection devices that promote sealing, such as rigid blisters, and is wrapped separately to prevent loss of the formulations and/or contamination. This element (01) is made of flexible material, that can be wrapped/rolled and does not cause inconvenience, allergic reactions or irritations when in contact with mucous and semi-mucous membranes, for example, but not limited to latex from natural rubber or other non-irritant rubbers or similar. Its sizes are between 1 and 2 cm, preferably 1.5 cm in diameter, and from 10 to 15 cm, preferably 12 cm in length when elongated. Both the diameter and the length of the packaging type encasement (01) may vary according to the purpose of use.

The portable applicator (08) presents a cylindrical format and it is covered at the moment of use by an anatomical encasement part (12), which in turn, is covered by the encasement (01). The portable applicator (08) is composed of two encasable parts, one upper (09) and the other lower (10), attached by means of a thread, Luer Lock system or any other encasement and locking system (11). The fact that the applicator is comprised of encasable two parts (09)(10) means it can be discretely carried to various locations, without generating embarrassment during application of the formulations because it is extended when the two parts are joined.

The applicator (08) is particularly useful for pushing the product contained in the compartment of the packaging on the desired site, mainly in the vaginal and rectal cavities, places where it is hard to put medications with the use of the fingers only. It can be disposable or otherwise, being washable and reusable after use, without risk of contamination, as this part does not come into contact with mucous and semi-mucous membranes. It is made of plastic material that is rigid enough to provide support at the moment of application and is non-irritant, although it does not have direct contact with the mucous membranes of the user's body where he/she wishes to apply the formulation. Its sizes are between 0.6 and 1.2 cm, preferably 0.8 cm in diameter and the two parts of which it is composed are 5 to 8 cm, preferably 6 cm in length each, amounting to 10 to 16 cm long, preferably 12 cm in length when extended for use.

The anatomic encasement part (12) is attached on the upper part of the portable applicator (08). Its distal end presents an anatomic form, designed to assist the output of the product from inside the packaging inwardly of the vagina or rectum, without causing injury to the user. At its lower part an opening is located for encasing the portable applicator (08). This part may be disposable or otherwise, since it will be covered by the encasement (01), and may be washed and reused after use. It is preferably made of rubber or plastic material, should be rigid enough to provide support at the moment of application and should also have minimal softness to promote comfort at the moment of application. Another possible configuration consists of a part injected into the rigid polymer and covered by a polymer/rubber layer to provide greater comfort to the user. It presents approximately 1 to 2 cm, preferably 1.5 cm in diameter and 1.5 to 2.5 cm, preferably 2 cm in length, and may present different sizes according to the purpose.

The following form of use is recommended to dispense formulations in the vaginal and rectal regions:

a. Encase the two parts (09)(10) of the portable applicator (08), making it elongated;
b. Encase the lower part of the polymeric/rubber part (12) on the upper part of the portable applicator already elongated;
c. Withdraw the packaging type encasement (01) from the blister;
d. Attach the packaging type encasement (01), containing the product, on the upper part of the polymeric/rubber part (12) and unroll the packaging type encasement (01) along the polymeric/rubber part (12) and the portable applicator (08);
e. Introduce the combination set into the desired cavity;
f. Hold the end of the packaging type encasement (01) at the distal part of the applicator (08) upon introducing into the vagina or rectum and push the applicator (08) to empty the distal portion of the encasement (01), so that the product is released through the orifices (06) inside the cavity;
g. Withdraw the combination from the cavity and remove the packaging type encasement (01) from the applicator (08);
h. Discard the packaging type encasement (01);
i. If desired, wash the portable applicator (08) and the polymeric/rubber part (12) for subsequent use.

For the oral, vulvar and anal regions, the following form of use is recommended:

a. Withdraw the packaging type encasement (01) from the blister;
b. Attach the encasement (01) containing the product at the tip of the user's finger and unwrap it along same finger, covering it;
c. Place the finger on the desired site and compress the reservoir against the wall of the site to be dispensed with the medicine;
d. Accordingly, the product will be released through the orifices (06), directly at the desired site;
e. If necessary, distribute the product to be dispensed, by gentle movements over the desired site;
f. Withdraw the finger from the site;
g. Unwrap/unroll the packaging type encasement (01) and discard it.

It is important to emphasize that the drawings and description set out do not have the weight of limiting the execution forms of the inventive concept now proposed, but rather to illustrate and make the conceptual innovations disclosed in this invention comprehensible. Therefore, the descriptions and pictures should be interpreted in an illustrative manner as opposed to a limitative one, and there may be other equivalent or analog forms of implementation of the inventive concept now disclosed and which does not stray from the scope of protection delineated in this invention.

The present specification referred to an innovative packaging which is attached either to an elongated applicator or to the user's finger, endowed with novelty, descriptive sufficiency [full disclosure], inventive step, industrial application and, consequently, satisfies all the essential requirements for the grant of the privilege sought.

1. An encasing device for dispensing topical products for oral, vulvar and anal uses, attachable to portable applicator for vaginal and rectal uses comprising a packaging type encasement device (01) having a compartment (02) filled with a desirable formulation in a single dose, wherein the compartment (02) is located in the upper portion of the encasement (01); said encasement (01) having orifices (06) located in its upper end and open lower end (03) where a finger or a portable applicator (08) is inserted for pressing the compartment (02); wherein said compartment upon compression expels the formulation through the orifices (06).

2. The encasing device for dispensing topical products for oral, vulvar and anal uses, attachable to portable applicator for vaginal and rectal uses wherein the encasement (01) comprises a small thimble (07) made of latex, malleable rubber or similar, holding the compartment (02) in the upper end (05) of the encasement (01).

3. The encasing device for dispensing topical products for oral, vulvar and anal uses, attachable to portable applicator for vaginal and rectal uses of claim 1, wherein said portable applicator is covered in its upper portion by an anatomical encasement part (12), said part (12) being covered by said encasement packaging (01).

4. The encasing device for dispensing topical products for oral, vulvar and anal uses, attachable to portable applicator for vaginal and rectal uses of claim 1, wherein said packaging type encasement (01) is disposable after use.

5. The encasing device for dispensing topical products for oral, vulvar and anal uses, attachable to portable applicator for vaginal and rectal uses of claim 1, wherein said packaging type encasement (01) remains in its unwrapped form until it is used without being compressed.

6. The encasing device for dispensing topical products for oral, vulvar and anal uses, attachable to portable applicator for vaginal and rectal uses of claim 1, wherein said packaging type encasement (01) is made of flexible material having a smooth surface.

7. The encasing device for dispensing topical products for oral, vulvar and anal uses, attachable to portable applicator for vaginal and rectal uses of claim 1, wherein the portable applicator (08) is composed of two parts (09)(10) and is encasable by means of thread, Luer Lock system or any other encasement and locking system (11).

8. The encasing device for dispensing topical products for oral, vulvar and anal uses, attachable to portable applicator for vaginal and rectal uses of claim 1, wherein the portable applicator (08) presents sizes between 0.6 and 1.2 cm, preferably 0.8 cm in diameter, and the two parts (09)(10) have 5 to 8 cm, preferably 6 cm in length each, reaching total length of 10 to 16 cm, preferably 12 cm, when extended for use.
